(19) United States
(12) Patent Application Publication
(10) Pub. No.: US 2020/0100725 A1
(43) Pub. Date: Apr. 2, 2020

(54) TOILET SENSE SYSTEM FOR HEALTH DIAGNOSTICS

(71) Applicants: Jared TEMANSON, Edgewater, CO (US); Jared LEIDICH, Edgewater, CO (US)

(72) Inventors: Jared TEMANSON, Edgewater, CO (US); Jared LEIDICH, Edgewater, CO (US)

(21) Appl. No.: 16/588,040

(22) Filed: Sep. 30, 2019

Related U.S. Application Data
(60) Provisional application No. 62/739,248, filed on Sep. 30, 2018.

(51) Int. Cl. A61B 5/00; A61B 5/20; A47K 17/00

(52) U.S. Cl. CPC A61B 5/48; A61B 5/208; A61B 5/0002; A61B 2562/0247; A61B 5/6887; A47K 17/00; A61B 2562/0257; A61B 5/0059

(57) ABSTRACT

A health diagnostics system includes an interne-of-things (IoT) toilet device adapted to be mounted on or within a toilet bowl. The IoT toilet device comprises one or more sensors for collecting measurements of human excrement residing within the toilet bowl and also includes an excrement analysis module stored in memory and executable by a processor to infer a property of a human bowel movement based on the collected measurements, where the inferred property includes at least one of color, volume, mass, consistency, and frequency

### BACKGROUND

Medical professionals have long known there are insights to be gained into an individual's health from their bowel movements and excrement (stool). To date, however, collecting those insights has depended on unreliable and uncomfortable conversations with patients. Doctors either rely on anecdotal information received conversationally from patients, or physical samples received intermittently. These methods do not provide medical professionals the credible, objective data they require to make meaningful diagnoses, or afford them the trend data necessary to track and manage responses to prescribed changes in diet and or a new regime of medication, conventional or experimental. Furthermore, emerging artificial intelligence (AI) and machine learning techniques are capable of gleaning insights from large data sets that can correlate identified properties of something such as a stool sample (human excrement) to an early stage health condition that may be treatable before the condition shows substantial symptoms

### SUMMARY

Implementations of the herein disclosed technology provide for device with a set of sensors for collecting data from a toilet bowl to infer properties pertaining to human excrement. In various implementations, the sensors may identify data on the color, volume, mass, consistency and frequency of the excrement produced by the bowel movement of an individual.

In one implementation, the device utilizes a set of sensors, an excrement analysis module, a health diagnosis module, and a user interface. The excrement analysis module aggregates data received from the set of sensors to provide a quantitative set of qualities associated with the user's excrement.

In another implementation, data produced by the excrement analysis module is transmitted to a health diagnosis module where the data may be used in various ways. For example, the data may be catalogued against existing diagnostics tools and those being further developed by medical professional. This distillation of data may also be trended and analyzed alongside other known medical conditions and or environmental circumstances—such as diet, stress, chronic disease, or medication.

In one implementation, the data collected by the disclosed system is analyzed in view of other aggregated data from similar devices to provide the user with a recommendation or medical diagnosis. Both the data collected by the system and the analytical inferences derived from such data may be useful to the user, their medical service provider, their insurer, and to the public. It is useful to the user as an indicator of trends in their bowel movements. The data could be useful to the broader public as a database of information including, for example, information about toilet activity from which correlations between the condition of fecal matter can be made to diseases, disorders or other trends in health. By correlating factors identified using the disclosed systems and techniques, medical experts could diagnose diseases or other troubling medical conditions more easily and more quickly. Further, by analyzing aggregated data collected from many different IoT toilet devices in different households across a population, the disclosed analytics system may be used to diagnose health problems and show paths to their cure.

In various implementations, the disclosed IoT toilet device may leverage several design approaches. The device may be installed in a plurality of contexts, from a private residence at the discretion of a private individual to a private establishment at the discretion of that private entity or a public space at the discretion of that public institution. Data from the sensor-enabled toilet or toilet application may provide utility in various ways to various audiences. For example, data collected by the IoT device may be used privately for individuals interested in monitoring general health or a medical condition, such as by receiving diagnostics indicating the whether the condition is worsening, improving, and/or suggestions on how to improve the condition. In still other implementations, the data collected by the disclosed IoT toilet device could be used by an insurance underwriter to audit or asses a potential insurer's health and wellness or by a medical professional to that same end.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Other implementations are also described and recited herein.

### BRIEF DESCRIPTION OF THE DRAWINGS

FIG. 1 shows an example smart toilet seat with no submerged sensors.

FIG. 2 shows another example smart toilet seat with no submerged sensors, viewed from the underside.

FIG. 3 illustrates an exploded view of an example smart toilet seat with no submerged sensors.

FIG. 4 illustrates an example clip-on smart toilet sensor with no submerged sensors.

FIG. 5 illustrates an exploded view of an example clip-on smart toilet sensor with no submerged sensors.

FIG. 6 illustrates another example clip-on smart toilet sensor with no submerged sensors, shown in a toilet.

FIG. 7 illustrates an example clip-on smart toilet sensor with submerged sensors.

FIG. 8 illustrates an example clip-on smart toilet sensor with submerged sensors in a toilet.

FIG. 9 illustrates an example smart toilet seat with submerged sensors.

FIG. 10 shows an example smart toilet assembly.

FIG. 11 illustrates an example non-load-bearing hinge that may be used for mounting an IoT toilet device.

FIG.12 is a block diagram illustrating exemplary aspects of a system that uses an IoT toilet device for generating health diagnostics and recommendations.

### DETAILED DESCRIPTION

Leveraging modern sensors, data communication, data analysis and storage technology could empower medical practitioners and data scientists to provide more passive, proactive health services. It could also empower health conscious consumers to make more proactive lifestyle changes. Incorporation of such technology into the delivery of medical services, and adoption by end users has to date been hindered by over-engineered and exceedingly complicated solutions without tried, tested, and actionable diagnostics regimes against which to juxtapose results.

The herein disclosed technology provides a system including a toilet-mounted device that includes one or more sensors for collecting measurements of human excrement residing within a toilet bowl. The system further includes a processor-executable excrement analysis module that analyzes the collected measurements to infer properties of a human bowel movement. According to one implementation, the excrement analysis module employs diagnostics tools like the Bristol Stool Chart (a.k.a. Bristol Stool Scale or the Bristol Stool Form Scale) to analyze the collected data. In one implementation, the system is configured to identify at least one potential health deficiency associated with the human excretion based on the inferred property and to initiate a remedial action based on the identification of the at least one potential health deficiency.

The herein disclosed technology includes a physical mechanism referred to herein as an IoT (internet of things) toilet device to sense toilet activity and to collect and analyze data from that activity.

In various implementations, the IoT toilet device is configured to measure or derive quantified values for one or more properties of a human bowel movement including: color or the user's excrement for each bowel movement, mass of the user's excrement for each bowel movement, volume of the user's excrement for each bowel movement, the consistency of a user's excrement for each bowel movement, and the frequency at which the user has bowel movements.

By measuring or deriving one or more of the above-listed properties and/or by performing analytics on such properties, the IoT toilet device is adapted to infer potential health deficiencies and, when applicable, promote remedial actions such as by providing the user with helpful insights on how to treat an underlying medical condition, address a deleterious lifestyle (e.g., diet, exercise), etc.

The first property of human excrement that could be measured or derived using this device is excrement color. Stool color may reflect how much bile is in a person's stool and may serve as a leading indicator of not only digestive health, but health generally. Stool is sometimes categorized according to six different color qualities: green, light-white or clay colored, yellow, black, bright red or orange. Depending on an individual's stool color, different root causes may be investigated, either health- or dietary-related. For example, black colored stool may indicate bleeding in the upper-gastrointestinal tract, where yellow stool may reflect excess fat and a malabsorption disorder, like celiac disease. Collecting trend data (i.e. data collected and analyzed over time) and coupling with other supporting anecdotal or measured data may make this metric supremely valuable to individuals and medical professionals.

The second property of human excrement that could be measured or derived using this device is excrement mass, or the mass of the excrement a user expels during a bowel movement. Similar to and along with stool consistency, mass could serve as a leading indicator of an individual's health and or dietary habits. Fecal impaction, for example, could be a serious problem that could lead to serious illness or death. Defined as a bowel condition in which hard, dry stool that can be stuck in an individual's rectum, mass, coupled with other inferred qualities—as well as qualitative qualities shared by a user or other IoT devices could suggest a multitude of lifestyle issues, including a low-fiber diet and inadequate fluid intake. It could also suggest an issue resulting from medication, such as a laxative that may be prescribed to a user.

The third property of human excrement that could be measured or derived using this device is excrement volume, or the volume of the excrement a user expels during a bowel movement. Volume could be an important indicator of health. Coupled with other metrics, like frequency, volume could be indicative of certain health concerns. For example, frequent bowel movements of modest volume can indicate left colon or rectal disease, where more voluminous stool could indicate small bowel or right colon disease. Trend data capturing volume of a user's bowel movements could also suggest critical bowel malabsorption concerns, critical to both dietary and or medicinal regiments that either a doctor would prescribe or to which an individual might choose to subscribe intentionally or inadvertently.

The fourth property that could be measured or derived using this device is frequency, or the frequency at which a user has a bowel movement. Individuals who experience frequent bowel movements, or a frequent urge to move their bowels, could have a larger health concern which medical professionals may be interested in. Irritable bowel syndrome (IBS) is one of many health issues that frequent bowel movements may indicate. Frequent bowel movements may also be symptomatic of celiac disease, diverticulitis, or a bacterial or viral infection. Frequent bowel movements may also reflect a negative reaction to surgery or medicinal regime and coupled with other stool qualities may help medical professionals diagnose the broader issue.

The fifth property of human excrement that could be measured or derived using this device is excrement consistency, or the consistence of the excrement a user expels during a bowel movement. Healthy stool could generally be regarded somewhere between a firm and soft consistency. If individual's bowel movements trend heavy in one direction of the other, this may suggest digestive or dietary-fiber issues. Consistency is a significant value in the Bristol Stool Chart. According to the Bristol Stool Chart, bowel movements with a consistency consistent with “Types 3 and 4” are ideal. On a scale of 1 to 7, this leaves 5 other “Types” that could indicate constipation or incontinence, either of which could indicate a broader dietary or chronic health concern, including Crohn's disease, biliary disease, and celiac disease.

According to various implementations, an IoT toilet device disclosed herein includes sensors to collect measurements of or relating to human excrement within a toilet bowl and logic (e.g., a processor and computer-executable instructions) to analyze the collected measurements and infer one or more of the five properties enumerated above on such basis.

There are several different types of sensors that could be useful in this analysis. In some implementations the IoT toilet device includes a load cell. The load cell could be used for multiple purposes. First, the load cell could establish that the device is being used and serve as a control trigger to command other sensors to begin collecting data.. Second, the load cell could distinguish one user from another by measuring their weight, or weight shift during toilet use. For example, the IoT device may be configured to store weight profiles associated with different uses and identify a current user based on the weight measured by the load cell. Third, a load cell could be used to establish the mass of excrement expelled during a bowel movement by measuring the change in weight of a user. Finally, a load cell could be used to measure the cadence, or rate of expulsion of excrement during a bowel movement. For example, if a user's mass changes very rapidly as a “step function” during a bowel movement, meaning the mass change is near instant from one mass to another, it could indicate solid stool in one discrete clump. If a user's mass changes fluidly, or continuously throughout a bowel movement it could indicate watery excrement.

In some implementations the IoT toilet device includes a proximity sensor to register fecal volume, such as by measuring a change in water level in the toilet. From this change in water level, a volume of excrement could be approximated. This sensor could also capture the cadence of a bowel movement by measuring the pace at which the water level changes. For instance, if the water level rises in spurts, as a step function, this would indicate chunks or logs of excrement. If the rate of change of the water level was continuous this would indicate watery excrement. Exemplary suitable proximity sensors included light-based sensors, capacitive sensors, and ultrasonic sensors.

In some implementations the IoT toilet device includes a color sensor to measure the color/shade of the user's bowel movement. This sensor could be a Red Green Blue sensor reporting the intensity of each light component before and after a bowel movement and subtracting away the light intensity of the common background to approximate the color of the excrement in the toilet.

In some implementations the IoT toilet device includes an infrared temperature sensor to collect the relative temperature of the user's bowel movement. This could measure the temperature of feces directly as it exits the anus or a change in water temperature that could be used to mathematically approximate the temperature of the feces prior to encountering the water.

In some implementations the IoT toilet device includes a physical thermometer to measure a user's body temperature. This could be affixed to the toilet seat and sense a user's body temperature when in contact with a user's skin when they are sitting on the seat. In some implementations the IoT toilet device includes a camera to take a picture of the user's excrement. The camera could point into the toilet bowl to take a picture of the excrement within the toilet bowl. A picture of the user's excrement could be used to derive properties of the excrement such as color, and consistency of the excrement. A picture of excrement could identify the presence of blood in stool or foreign objects. The camera could capture still pictures or videos.

In some implementations the IoT toilet device could include one or more additional sensors including within limitation a salinity sensor, a sensor that measures the conductivity of the toilet water, a sensor that measures the PH of the toilet water, a sensor that measures the methane present in the air above the toilet water, a sensor that measures the glucose in the toilet water or in a person's stool or urine, a sensor that measures estrogen levels, a sensor that measures testosterone levels, a sensor that measures the details of blood, a sensor that measures serotonin, a sensor that measures adrenaline, a sensor that measures enzymes, a sensor that measures cortisol, a sensor to detect different kinds of bacteria and any other sensor that can glean insight into body chemistry or condition from a toilet event. Some examples of demographics that may be interested in using this device may include:

1. People with Chron's disease

2. People with celiac disease

3. People with Irritable Bowel Syndrome

4. People with regular blood in their stool

5. People with colon cancer

6. People with weak stomachs

7. People interested in correlating eating habits with digestive health

8. People interested in correlating lifestyle habits with digestive health

9. People who are interested in proving good digestive health to a healthcare provider

10. People with a general interest in their health conditions

In the following descriptions, the term “sensors” is used generally and may be understood as referring to one or more specific types of sensors listed above. In the following description, different example embodiments are described. It should be understood that the various sensors and features described with respect to any one embodiment may be suitable for integration within any other embodiment in lieu of or in addition to the specific features of that other embodiment that are shown and described herein.

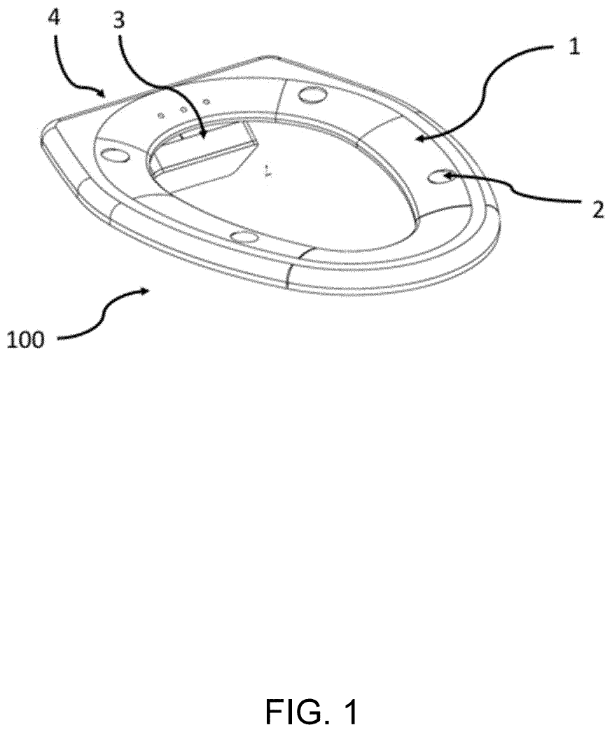

FIG. 1 shows an example IoT toilet device 100 that includes various sensors (e.g., a temperature sensor 2) and other electronics integrated within a smart toilet seat. Unlike other implementations disclosed herein, the IoT toilet device 100 does not include submerged sensors. The IoT toilet device 100 has some or all the included sensors affixed to a toilet seat. A toilet seat 1 with attached sensors could be used as a drop-in replacement for an existing toilet seat. Using a toilet seat with integrated sensors allows for unique functionality including integrated load cells for measuring a person's weight and change in weight during a bowel movement. This could be accomplished by measuring the change in a person weight during that toilet activity. A seat can also provide integrated temperature sensors directly in contact with a person's skin to read body temperature. A toilet seat Error! Reference source not found. may be integrated with temperature sensor(s) 2 and an electronics package 3 with several additional sensors (described below). The seat could be integrated with unique hinges 4 that support the seat but that do not hold the weight of the user, allowing for more accurate weight readings. The primary electronics package for this embodiment of the invention could include several sensors including, but not limited to, an ultrasonic distance sensor, an infrared temperature sensor, a color sensor, a camera, a gesture sensor, a light sensor, or any other sensor used to sense and monitor toilet activity. The primary electronics package could also include a microprocessor or microcontroller to manage, process, store, or transmit the data values collected by the sensors. The electronics package 3 could also include electronics used for wireless communication including but not limited to a wireless local area network, Bluetooth, or a direct wireless data link to a wireless modem.

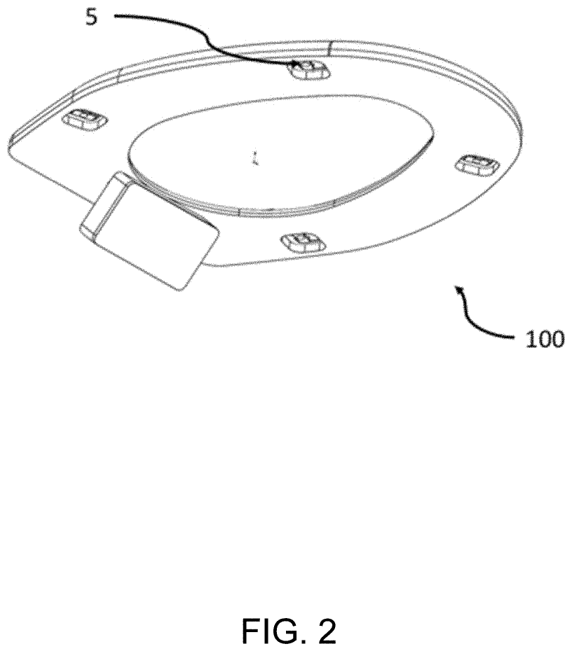

FIG. 2 illustrates an alternate view of the IoT toilet device 100 illustrated in FIG. 1. In this view, load cells 5 are visible on the underside of the toilet seat. As discussed above, the load cells 5 may be adapted to measure a person's weight and the change in weight of that person during a bowel movement to infer a mass of the human excrement collected in the toilet bowl.

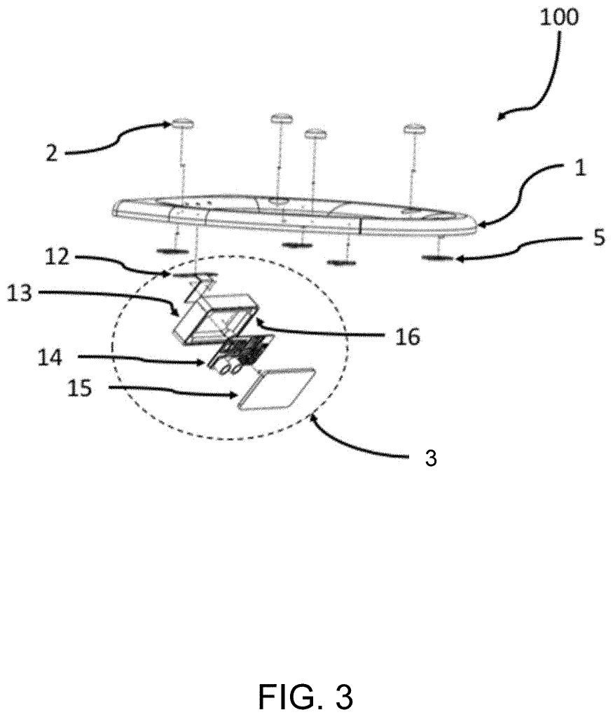

FIG. 3 illustrates an exploded view of the IOT toilet device 100 illustrated in FIG. 1. This view better illustrates the position of load cells 5 as well as temperature sensors 2, which are shown embedded in the seat 1 such that they may be in direct contact with a user's skin. The electronics package 3 is expanded to illustrate various internal and external components including a clip 12 for attaching an electronics box 13 to the toilet seat 1. The electronics box 13 houses a printed circuit board 14 as well as one or more (not shown) such sensors, such as one or more color sensors, proximity sensors, infrared temperature sensors, cameras, or video cameras, which could each be integrated within the housing and electrically coupled to the printed circuit board. The electronics package 3 further includes a seal 16 and a faceplate 15 for creating a watertight enclosure allowing the unit to be washed off directly.

Although not shown, it may be understood that the electronics package 3 further includes transmission control electronics for transmitting collected sensor data across a wired or wireless network. In some implementations, the electronics package includes a processor and memory storing computer-executable instructions (e.g., software or firmware) for detecting user events (e.g., user presence, bowel movement), commanding sensors to collect data, and/or locally analyzing the collected data to infer one or more properties of a human bowel movement. For example, the memory may store an excrement analysis module that analyzes collected sensor data to infer a property of a bowel movement such as color, volume, mass, consistency, and/or frequency. In still other implementations, the memory stores a health diagnosis module that performs further analytics, such as by comparing the inferred bowel movement property to reference data to identify a health deficiency and/or to initiate a remedial action such as to inform a user of a determined insight, to provide a health recommendation, etc. In some implementations, some or all software operations described herein as performed by the IoT device are performed remotely, such as by a cloud-based server in communication with the device.

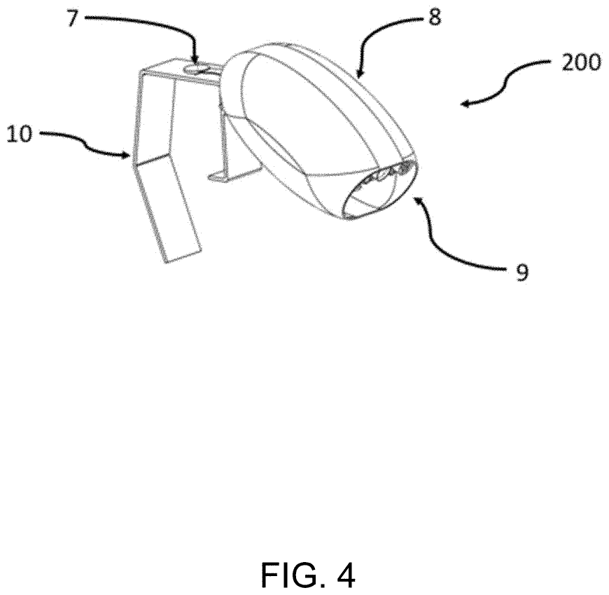

FIG. 4 illustrates another example IoT device 200 referred to herein as the “Clip-on” design. In contrast to FIG. 1-3, the IoT device 200 is a structure entirely separate from the toilet seat. Like the implementation of FIG. 1-2, this design does not have any sensors that are designed to be submerged within the liquid of the toilet bowl. In this design, a sensor package is housed in a shell 8. This sensor package may be understood as including one or more of the various sensors described elsewhere herein. The shell 8 is shown attached to a mounting clip 10 which is usable to affix the IoT device 200 to a toilet. By example and without limitation, the IoT device 200 is shown to include a force measuring device 7 that sends a signal to control electronics within the shell 8 to initiate a sensing event. In implementations where the force measuring device 7 is capable of measuring user weight with sufficient precision, the force measuring device 7 may be used to differentiate one user of a device from another. The IoT device 200 may include various features designed to provide modularity in several ways. For example, the arm portion of the mounting clip 10 may be made of different mediums of varying pliability. Greater pliability of the arm could allow users to secure the present invention to the rim of any toilet. To ensure modularity, the mounting clip 10 may also incorporate a mechanical fastener that allows users to secure the Clip-on embodiment to a toilet and to remove and relocate as desired. This may be accomplished by hand or using the assistance of tools and adjust the angle at which the shell 8 is set.

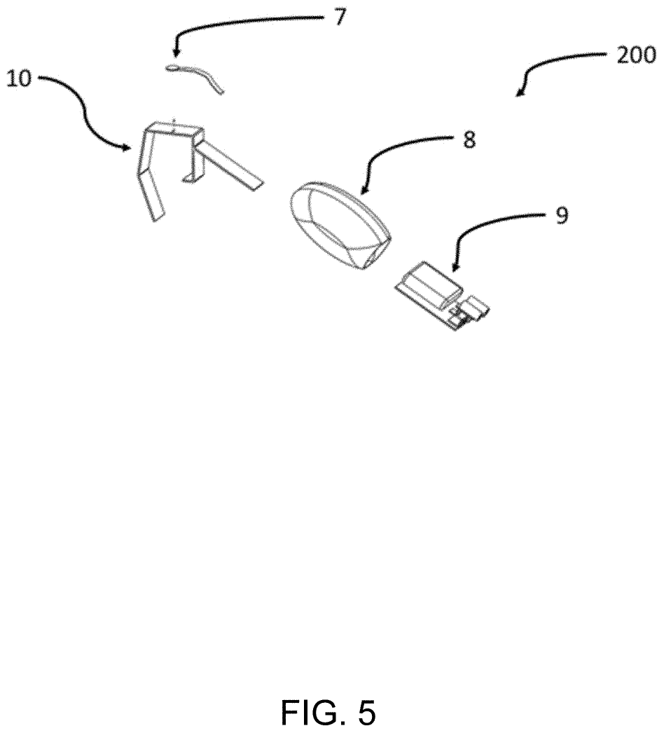

FIG. 5 illustrates an exploded view of the IoT device 200 referred to herein as the “Clip-on” design. This view shows the mounting clip 10, force measuring device 7, and a printed circuit board 9 that supports one or more sensors, such as color sensors, proximity sensors, infrared temperature sensors or cameras or video cameras, all of which may be integrated within or on the shell 8.

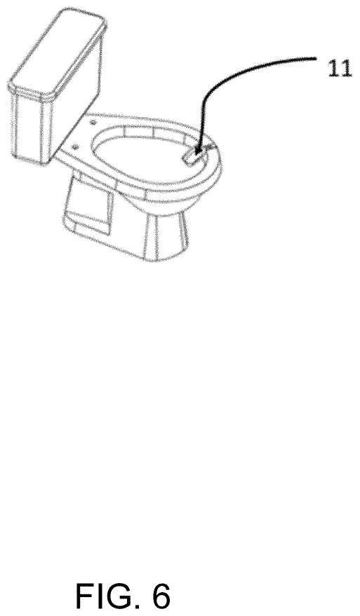

FIG. 6 illustrates an alternate view in which the IoT device 200 of FIG. 4-5 is shown mounted in a toilet. Other aspects of the IoT toilet device 200 not described in detail may be the same or similar to those described elsewhere herein.

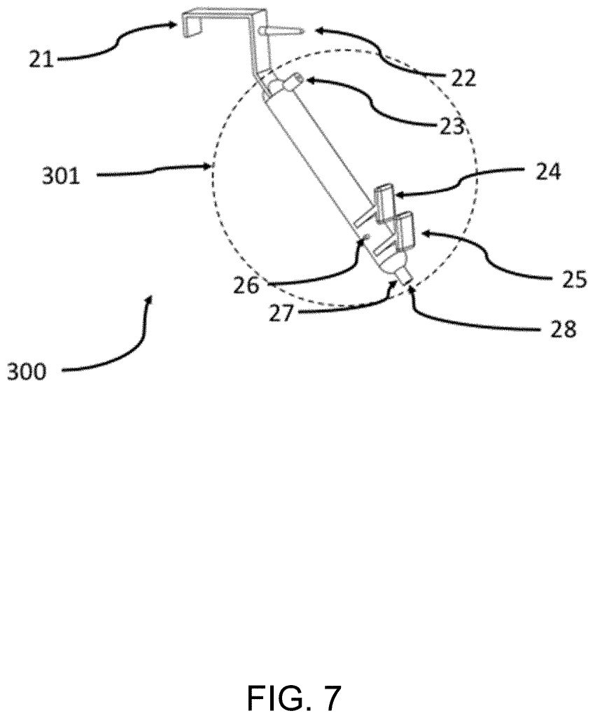

FIG. 7 illustrate another example IoT device 300 referred to herein as the “Submerged Clip-on” design. In this implementation, a clip 21 secures the device to the side of a toilet with a sensor package 301 that is designed to be at least partially submerged in toilet water. In one implementation, the wireless data transmission mechanism such as wifi or Bluetooth (an antenna 22 is shown) is coupled to control electronics (not shown) adapted to transmit data collected by the sensor package 30 across a network such as the internet or other platform to one or more servers where data is stored. Among other sensors, the IoT device 300 includes an ambient pressure sensor 23 and a submerged pressure sensor 28 that may be usable to determine a current water level in a toilet and thereby approximate volume of the excrement in a bowel movement. The IoT device 300 additionally includes an infrared temperature sensor 24 and a direct thermometer 27. The infrared temperature sensor 24 is usable to approximate fecal or urine temperature and body temperature while the direct thermometer is usable to approximate temperatures based on changes in water temperature. A probe 26 could detect things like salinity of the water after a toilet event.

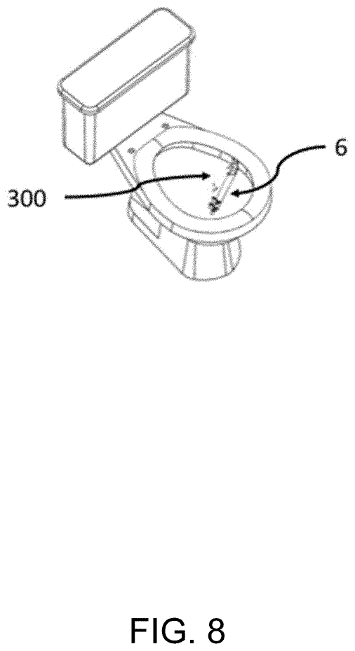

FIG. 8 illustrates an alternate view of the IoT device 300 referred to herein as the “Submerged Clip-on” design. The IoT device 300 may include various features that provide modularity in several ways. For example, the clip 21 may be made of different mediums of varying pliability. Greater pliability of the clip 21 could allow users to secure the present invention to the rim of any toilet, where the rim of the toilet is the surface that resides below the seat when the seat is down. To ensure modularity, the clip 21 may also incorporate a mechanical fastener that allows users to secure the Clip-on embodiment to a toilet and to remove and relocate as desired. This may be accomplished by hand or using the assistance of tools and adjusting the angle at which the housing is set. The sensor assembly to which all or some of the sensors are affixed may also be separate or separated from the device that connects to the toilet. The point at which the sensor assembly integrates with the device that connects to the toilet may operate on a hinge to allow the user the ability to adjust the angle of the rod. The rod may also possess a telescoping functionality to allow users to adjust the length.

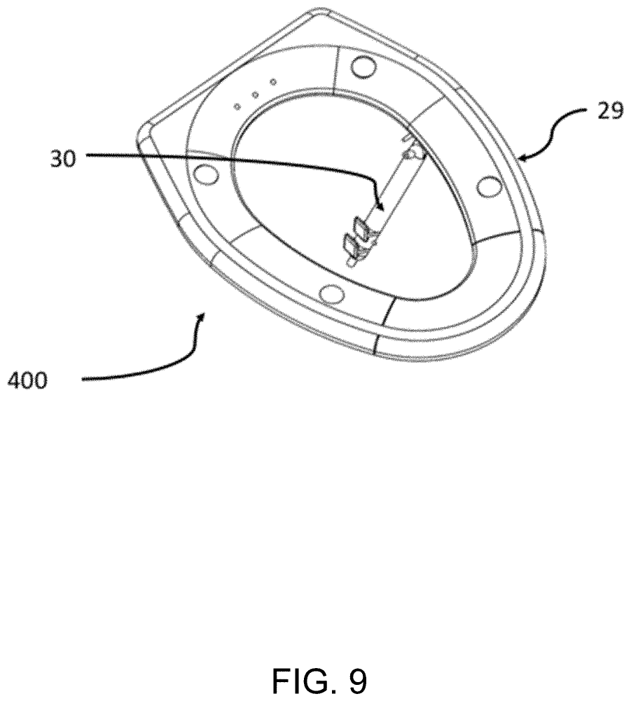

FIG. 9 illustrates still another example IoT device 400 referred to herein as the “Submerged Fixed” design. This IoT device 400 utilizes a sensor assembly 30 that is fixed to a toilet seat 29. This IoT device 400 can infer properties of human excrement based on data collected by sensors embedded in the toilet seat 29 and sensors attached to a rod 30. The rod 30 is designed to be at least partially submerged in toilet water during normal use and may include one or more sensors at the distal end of the rod that is submerged in the water as well as one or more sensors more proximate to the opposite end of the rod that may not be submerged in water.

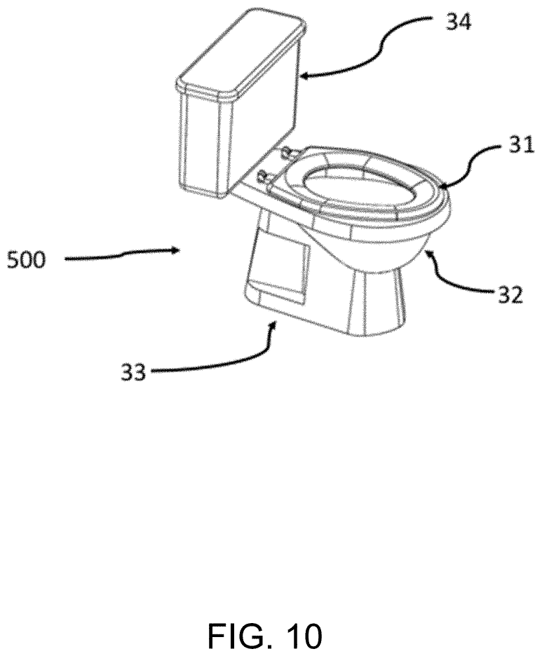

FIG. 10 illustrates an example IoT device 500 referred to herein as the “Sensing Toilet” design. This IoT device 500 could include a sensing toilet seat 31. A sensing toilet could include sensors in the outgoing plumbing of the toilet 33 that could monitor the substances exiting the toilet 33. A sensing toilet could additionally include a sensing package in the toilet tank 34 that could monitor things like toilet usage. This sensor package could monitor which flush option was chosen for toilets with multiple flush options like a low volume flush for urine or a higher volume flush for a bowel movement. This data could be used to establish, or gather extra data surrounding frequency of bowel movements for a user. It provides an additional data point that could be used in the excrement analysis module to verify or refute that an event was a bowel movement (as opposed to a urination event).

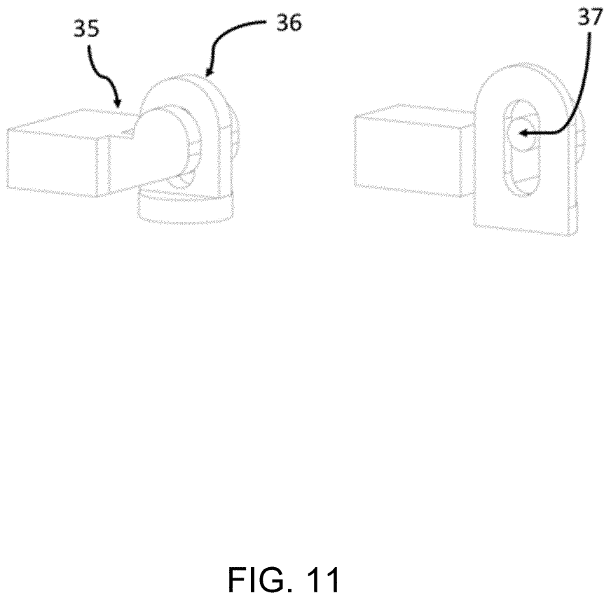

FIG. 11 illustrates a non-load-bearing hinge. A hinge such as the one shown could be used to ensure that the hinges holding a toilet seat down does not carry a substantial load from the user affecting the load cell measurements collected by load cell sensors described with respect to other implementations herein.

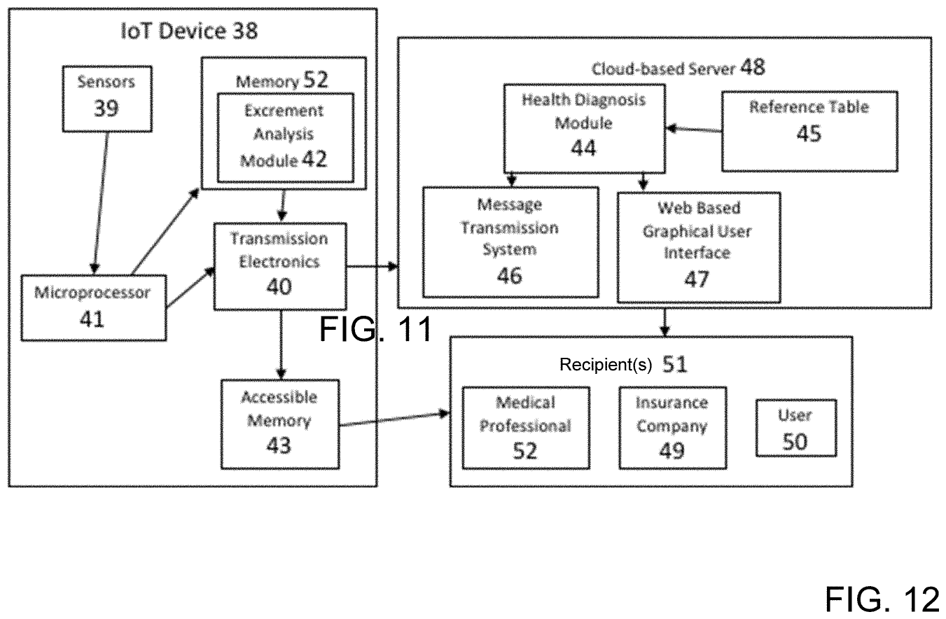

FIG. 12 illustrates an exemplary health diagnostics system 1200 that performs both toilet sensing and analytics on collected sensor data to infer various properties of human bowel movements, identify potential health deficiencies and/or health insights, and provide remedial actions. The system 1200 includes an IoT device 38 that includes sensors 39 that collect data on a user's excrement. The IoT device includes memory 52, such as volatile or non-volatile memory, and a microprocessor 41. The memory 52 stores an excrement analysis module 42 that analyzes collected sensor data to derive properties of a human bowel movement such as excrement color, volume, mass, consistency and frequency. The excrement analysis module 42 could either store that information in on-board memory 43, or transmit it to the internet via transmission electronics 40. In either case, such information could be accessed by a user 50 using a data transmission system such as blue tooth. In some implementations, the excrement analysis module 42 is distributed across one or more devices and some operations may be performed either locally within the IoT device 28 (as described above) while others are performed remotely (e.g., on a cloud-based server).

The herein disclosed system may, in some implementations, include a cloud-based server 48. A cloud-based server is a computer or server located in remote location that is intended for use only through the internet. Data sent to a cloud-based server 48 may be analyzed by a health diagnosis module 44 (e.g., computer-executable instructions stored in memory). The health diagnosis module 44 may, for example, utilize an existing reference table 45 such as the Bristol stool chart, and other tools developed by medical professionals for analyzing stool to identify a health deficiency or to draw other conclusions about a person's excrement or health. For example, the health diagnosis module 44 may determine that the user is developing a certain health condition like Chron's disease and/or provide the user with recommendations pertaining to changes in diet or lifestyle. In one implementation, the health diagnosis module 44 receives properties inferred from a human bowel movement (e.g., from the excrement analysis module 42), compares the inferred properties to data in the reference table 45 to autonomously identify at least one potential health deficiency associated with the human excrement. Based on the identified potential health deficiency, the health diagnosis module generates suggested remedial actions. For example, the health diagnosis module 44 could use an existing medical chart like the Bristol stool chart, reference data from the excrement analysis module 42, diagnose a person as constipated, and generate a suggestion for a remedial action, such as eating more fiber. The device could also identify positive trends in health based on insights gathered. This information could be displayed on a Web-based Graphical User Interface 47 online where a user could view it, or could be transmitted via a message transmission system 46 to a recipient 51 that could be a user 50, a medical professional 52, an insurance company 49, or any other person or entity that could utilize the data for any reason. In a different implementation of the IoT device 39, the microprocessor 41 could perform the same functions as the cloud based server 48, and communicate the results directly to a user 50 without necessitating the data be transmitted through the internet. In yet another implementation of the device the raw data from the sensors could be transmitted to a cloud based server 48 and the functions of the microprocessor 41 could be performed at the cloud based server.

The implementations described herein are implemented as logical steps in one or more computer systems. The logical operations may be implemented (1) as a sequence of processor-implemented steps executing in one or more computer systems and (2) as interconnected machine or circuit modules within one or more computer systems. The implementation is a matter of choice, dependent on the performance requirements of the computer system being utilized. Accordingly, the logical operations making up the implementations described herein are referred to variously as operations, steps, objects, or modules. Furthermore, it should be understood that logical operations may be performed in any order, unless explicitly claimed otherwise or a specific order is inherently necessitated by the claim language.

What is claimed is:

1. A system comprising:
an internet-of-things (IoT) toilet device adapted to be mounted on or within a toilet bowl, the IoT toilet device comprising one or more sensors for collecting measurements of human excrement residing within a toilet bowl; and
an excrement analysis module stored in memory and executable by a processor to infer a property of a human bowel movement based on the collected measurements, the inferred property including at least one of color, volume, mass, consistency, and frequency.

2. The system of claim 1, wherein the IoT toilet device further comprises transmission control electronics to transmit the inferred property to a cloud based server.

3. The system of claim 1, further comprising:
a health diagnosis module stored in memory and executable by a processor to:
compare the inferred property to reference data to identify at least one potential health deficiency associated with the human excrement; and
initiate a remedial action based on the identification of the at least one potential health deficiency.

4. The system of claim 1, wherein the one or more sensors includes a proximity sensor and the excrement analysis module is further executable to infer a volume of human excrement present in the toilet bowl based on a detected change in water level within the toilet bowl detected by the proximity sensor.

5. The system of claim 1, wherein the excrement analysis module is still further executable to infer at least one of a cadence associated with an individual bowel movement for an individual user and a frequency of bowel movements for the individual user.

6. The system of claim 1, wherein the one or more sensors further include a weight sensor and wherein the excrement analysis module is further adapted to:
analyze weight data collected by the weight sensor to determine a change in user mass as a result of the human bowel movement; and
based on the determined change in user mass, infer a mass of the human excrement collected in the toilet bowl.

7. The system of claim 1, wherein the one or more sensors includes at least one of a color sensor that records a color of the human excrement and an infrared sensor that records a temperature of the human excrement.

8. The system of claim 1, wherein the IoT toilet device includes a mounting clip that secures around a rim of the toilet, the mounting clip being adapted to rest between the rim of the toilet and a toilet seat.

9. The system of claim 8, wherein the IoT toilet device further includes a rod portion that extends from the mounting clip, the rod portion having a distal end supporting at least one of the one or more sensors that are submerged in toilet water when the mounting clip is secured to the rim of the toilet seat.

10. The system of claim 9, wherein one of the sensors supported by the distal end of the rod portion collects a measurement of at least one of salinity, acidity or blood content.

11. A method comprising:
collecting measurements of human excrement residing within a toilet bowl at one or more sensors of an IoT toilet device mounted at least partially within a toilet bowl; and
analyzing the measurements with a processor to infer a property of a human bowel movement based on the collected measurements, the inferred property including at least one of color, volume, mass, consistency, and frequency.

12. The method of claim 11, further comprising:
transmitting the inferred property to a cloud based server.

13. The method of claim 11, further comprising:
identifying at least one potential health deficiency associated with the human excretion based on the inferred property; and
initiating a remedial action based on the identification of the at least one potential health deficiency.

14. The method of claim 11, wherein the one or more sensors of the IoT device include a proximity sensor and analyzing the measurements to infer the property further comprises:
inferring a volume of human excrement present in the toilet bowl based on a detected change in water level within the toilet bowl detected by the proximity sensor.

15. The method of claim 11, wherein analyzing the measurements to infer the property further comprises:
inferring at least one of a cadence associated with an individual bowel movement for an individual user and a frequency of bowel movements for the individual user.

16. The method of claim 11, wherein the one or more sensors further include a weight sensor and the method further comprises:
analyze weight data collected by the weight sensor to determine a change in user mass as a result of the human bowel movement; and
based on the determined change in user mass, infer a mass of the human excrement collected in the toilet bowl.

17. The method of claim 11, wherein the one or more sensors includes at least one of a color sensor that records a color of the human excrement and an infrared sensor that records a temperature of the human excrement.

18. The method of claim 11, wherein the IoT toilet device includes a mounting clip that secures around a rim of the toilet, the mounting clip being adapted to rest between the rim of the toilet a toilet seat.

19. The method of claim 18, wherein the IoT toilet device further includes a rod portion that extents from the mounting clip, the rod portion having a distal end supporting at least one of the one or more sensors that are submerged in toilet water when the mounting clip is secured to the rim of the toilet seat.

20. An IoT toilet device comprising:
a mounting clip that secures around a rim of the toilet, the mounting clip being adapted to rest between the rim of the toilet and a toilet seat;
a housing component attached to mounting clip and adapted to rest at least partially within a bowel of the toilet;
one or more sensors attached to the housing component and adapted to collect measurements of human excrement residing within a toilet bowl, the measurements being usable to infer at least one of color, volume consistency and frequency; and
transmission control electronics to transmit the measurements to a remote server.
